(19) United States
(12) Patent Application Publication
(10) Pub. No.: US 2010/0087676 A1
(43) Pub. Date: Apr. 8, 2010

(54) ENVIRONMENTALLY BENIGN AND SIMPLIFIED METHOD FOR PREPARATION OF AROMATIC DICARBOXYLIC ACID

(75) Inventors: Jae Sung Lee, Gyeongsangbuk-do (KR); Milan Hronec, Slovakia (SK); Kyung Hee Lee, Gyeongsangbuk-do (KR); Jin Won Kwak, Incheon (KR); Young Hwan Chu, Ulsan (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG PETROCHEMICAL CO., LTD., Ulsan (KR)

(21) Appl. No.: 12/531,038

(22) PCT Filed: Mar. 7, 2008
(86) PCT No.: PCT/KR2008/001297

§ 371 Date: Sep. 11, 2009

(30) Foreign Application Priority Data
Mar. 15, 2007 (KR) 10-2007-0025570

(51) Int. Cl. C07C 51/265; C07C 51/215

(52) U.S. Cl. 562/412; 562/409

(57) ABSTRACT

Disclosed is an environmentally benign and simplified method for preparing aromatic dicarboxylic acid. In the disclosed method, a mixed solvent composed of aromatic mono-carboxylic acid and water, instead of conventionally used low molecular weight carboxylic acid such as acetic acid, is used as a reaction solvent for an oxidation process; manganese and a small amount of transition metal element are used as catalysts; and carbon dioxide is used as a reaction stabilizer. Accordingly, it is possible to improve the yield and selectivity of the aromatic dicarboxylic acid.

### TECHNICAL FIELD

The present invention relates to an environmentally benign and simplified method for preparing aromatic dicarboxylic acid, which can improve the yield and selectivity of the aromatic dicarboxylic acid by using a mixed solvent including aromatic mono-carboxylic acid and water, instead of conventionally used low molecular weight carboxylic acid such as acetic acid, as a reaction solvent during an oxidation process, using manganese and a small amount of transition metal element as a catalyst, and using carbon dioxide as a reaction stabilizer.

### BACKGROUND ART

In general, aromatic dicarboxylic acid is a useful compound used as a raw material for a wide range of products. Terephthalic acid (TA), one aromatic dicarboxylic acid, is used as a main material for polyethylene terephthalate (PET), polyester fiber, and a polyester film for packaging and containers. The preparation of TA worldwide is more than 50 million tons per year, and the preparation of TA in one factory can be more than 100,000 to 800,000 tons per year.

Aromatic dicarboxylic acid, for example, terephthalic acid (TA) may be prepared through an exothermic oxidation reaction of an aromatic feedstock compound, for example, para-xylene (PX), by using air or other oxygen molecule sources as an oxidizer, and by using one or more heavy metal compounds and one or more reaction initiator compounds.

A method of oxidizing an aromatic feedstock compound by using such a liquid-phase oxidation reaction has been widely known to those skilled in the art. For example, U.S. Pat. No. 2,833,816 (by Saffer et al.) discloses a method of oxidizing an aromatic feedstock compound to a corresponding aromatic carboxylic acid.

The core of the method is to use a liquid-phase reaction using low-molecular weight carboxylic acid, such as acetic acid (HAC), as a major part of a reaction solvent. As the progress of the reaction, water is produced as a by-product, and at the same time, carbon monoxide and carbon dioxide are produced by partial combustion of the solvent and the aromatic feedstock compound.

When bromine is used as a reaction initiator, methyl bromide may be produced. When air is used as a source of providing oxygen molecules, gas emitted from a reaction include nitrogen gas and unreacted oxygen gas as well as carbon monoxide, carbon dioxide, and methyl bromide.

The heat generated from the oxidation reaction may be efficiently removed by vaporizing a mixed solvent of water and low-molecular weight carboxylic acid, condensing the vaporized solvent in at least one upper condensing device, and then recycling again the condensed solvent into a reactor.

Herein, in order to regularly maintain water concentration in the reactor, a part of the condensed solvent has to be purged to the outside of the reactor. Since there exist both water and low-molecular weight carboxylic acid in the condensates, the purged condensates must be separated into water and low-molecular weight carboxylic acid by using a separation system such as a distillation column, and then, only the low-molecular weight carboxylic acid must be recovered again.

Uncondensed emission gas requires additional oxidation devices in order to preferentially remove environmentally harmful materials such as methyl bromide. After passing through such a device, the emission gas includes only environmentally harmless materials, and then runs through an expander or a turbine. Accordingly, a part of a very large amount of energy included in the emission gas may be recovered as electrical power.

The above described oxidation reaction system guarantees the yield of 95% or more in a reaction, and has very excellent selectivity by minimizing the amount of by-products, such as aromatic mono-carboxylic aldehyde and aromatic mono-carboxylic acid, produced during the reaction. Therefore, the reaction system has been adopted for most processes of preparing commercial aromatic dicarboxylic acid.

However, in spite of the above described advantages, the conventional oxidation reaction system has various problems to be solved from the standpoint of economic efficiency and environmental safety.

One of the main problems is that bromine is used as a reaction initiator. Bromine plays an important role for initiating and accelerating an oxidation reaction, but causes a variety of corrosions in equipments. Accordingly, the available material is limited to a special corrosion resistance material such as titanium, and the equipments are required to be periodically changed.

Furthermore, the use of bromine will have many limitations in the future, because bromine is very harmful to the human body, thereby causing fatal results by contact with even an extremely small amount, and can worsen an atmosphere around a work place and cause serious environmental pollution.

Another main problem of the conventional system is that a low-molecular weight carboxylic acid, for example, acetic acid, is used as a reaction solvent. In the case of acetic acid, compared to water, the probability of producing a complex between a heavy metal catalyst and a reaction initiator is high, thereby helping catalysis of a reaction. However, due to burning during an oxidation reaction, a part of acetic acid is lost as carbon monoxide and carbon dioxide, and another part of that is converted into methyl acetate (MA) of high volatility. Therefore, some of acetic acid cannot sufficiently function as a solvent, and has disadvantage in economical aspect.

Actually, in the commercialization process, except for cost of aromatic feedstock, acetic acid occupies the largest portion of overall variable cost. Also, since the difference in boiling points between acetic acid and water (a by-product of a reaction) is not very large, large amounts of acetic acid is always vaporized together with water in an oxidation reactor, and thus a huge amount of energy is consumed in order to separate/recover acetic acid from water.

Besides, the addition of many devices for recovering acetic acid from a process causes an overall process to be complicated.

Acetic acid is a material that should not only be recovered as a solvent but also removed in advance when crude aromatic dicarboxylic acid is purified via hydrogenation process. Thus, the oxidation process and the purification process must be rigorously separated.

Acetic acid also corrodes equipments when used at high temperatures like bromine. Therefore, an expensive corrosion-proof material is required for the process, and this is disadvantageous factor from viewpoint of capital cost.

In the health, environment, safety aspects, acetic acid is also very harmful to the human body, and can pollute an environment of a work place by causing a bad smell. Accordingly, there is high probability that use of acetic acid is limited in the future.

Therefore, a new process of preparing aromatic dicarboxylic acid which is environmentally benign, more simplified and economically efficient is absolutely required.

### DISCLOSURE

### Technical Problem

The present inventor provides an oxidation reaction system using a new solvent and catalyst system, instead of using an economically/environmentally disadvantageous conventional reaction initiator and solvent, which is environmentally benign, and at the same time, guarantees the economical efficiency by the simplification of a process and the significant decrease of capital cost.

In the present invention, in order to solve the problems caused by using low-molecular weight carboxylic acid solvent such as acetic acid, a mixed solvent composed of water and aromatic mono-carboxylic acid is used for oxidation reaction. Therefore, the increase in variable cost and capital cost is suppressed, and environment of a work place is improved.

Also, in the present invention, a catalyst system organized by manganese and a small amount of transition metal element, excluding bromine (which is a conventionally used reaction initiator) is used as a reaction catalyst. Therefore, the increase in capital cost caused by bromine is suppressed, and environment of a work place is improved.

Also, in the present invention, carbon dioxide generated from an oxidation process is recovered, and is used for a reaction stabilizer for an oxidation reaction, thereby suppressing side reactions during a process of preparing aromatic dicarboxylic acid.

### Technical Solution

According to an aspect of the present invention, there is provided a method of preparing aromatic dicarboxylic acid, the method including: an oxidation process of preparing crude aromatic dicarboxylic acid by liquid-phase oxidizing aromatic feedstock compound; and a purifying process of removing impurities by hydrogenation of the crude aromatic dicarboxylic acid, wherein the oxidation process uses a mixed solvent including water and aromatic mono-carboxylic acid as a reaction solvent.

In an oxidation reaction system according to the present invention, when converting an aromatic feedstock compound into aromatic dicarboxylic acid through an exothermic liquid-phase oxidation reaction, a mixed solvent including aromatic mono-carboxylic acid and water, instead of low-molecular weight carboxylic acid, is used as a solvent.

Herein, an aromatic feedstock compound used as a raw material is an aromatic compound having oxidation-susceptible substituents that can be oxidized to a carboxylic group. For example, the oxidation-susceptible substituent may be an alkyl group, such as a methyl group, an ethyl group, or an isopropyl group.

In the aromatic feedstock compound, the aromatic may be a benzene nucleus, or a bicyclic/polycyclic nucleus, such as a naphthalene nucleus. It is preferable that the aromatic feedstock compound is an aromatic dialkyl compound that the number of oxidation-susceptible substituents on the aromatic is two.

Accordingly, examples of the aromatic feedstock compound include, but are not limited to, ortho-xylene(o-xylene), meta-xylene (m-xylene), para-xylene (p-xylene), 1-ethyl-4-methyl benzene, 1-ethyl-3-methyl benzene, 1-ethyl-2-methyl benzene, 1,4-diethylbenzene, 1,3-diethylbenzene, 1,2-diethylbenzene, 1-isopropyl-4-methyl benzene, 1-isopropyl-3-methyl benzene, 1-isopropyl-2-methyl benzene, 1-isopropyl-4-ethyl benzene, 1-isopropyl-3-ethyl benzene, 1-isopropyl-2-ethyl benzene, 1,4-diisopropyl benzene, 1,3-diisopropyl benzene, 1,2-diisopropyl benzene, 2,6-dimethyl naphthalene, 2,6-diethyl naphthalene, 2,6-diisopropyl naphthalene, 2-methyl-6-ethyl naphthalene, 2-methyl-6-propyl naphthalene, 2-ethyl-6-propyl naphthalene, etc.

In the oxidation of the aromatic feedstock compound, para-xylene produces terephthalic acid, meta-xylene produces isophthalic acid, and 2,6-dimethyl naphthalene produces 2,6-naphthalene dicarboxylic acid.

Aromatic mono-carboxylic acid, which is used as a solvent with water, must be oxidative intermediate of the aromatic feedstock compound. For example, when para-xylene is used as an aromatic feedstock compound, para toluic acid should be used as a solvent; when meta-xylene is used as an aromatic feedstock compound, meta toluic acid should be used as a solvent; and when 2,6-dimethyl naphthalene is used as an aromatic feedstock compound, 2-methyl-6-carboxylic naphthalene should be used as a solvent.

In the present invention, aromatic mono-carboxylic acid may be a compound selected from the group including a compound represented by Formula 1 and a compound represented by Formula 2.

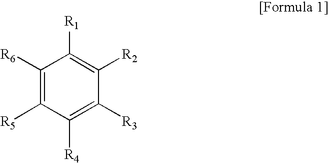

In Formula 1, from R₁ to R₆, one substituent is an alkyl group selected from the group including methyl, ethyl and isopropyl, another substituent is a carboxylic group, and the other substituents are hydrogen.

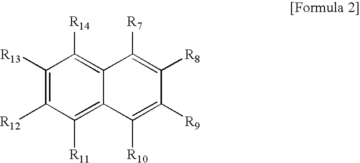

In Formula 2, from R₇ to R₁₄, one substituent is an alkyl group selected from the group including methyl, ethyl and isopropyl, another substituent is a carboxylic group, and the other substituents are hydrogen.

More preferably, aromatic mono-carboxylic acid may include one material selected from the group including para-toluic acid, ortho-toluic acid, meta-toluic acid, 2-ethyl benzoic acid, 3-ethyl benzoic acid, 4-ethyl benzoic acid, 2-isopropyl benzoic acid, 3-isopropyl benzoic acid, 4-isopropyl benzoic acid, 2-methyl-6-carboxylic naphthalene, 2-ethyl-6-carboxylic naphthalene, and 2-isopropyl-6-carboxylic naphthalene.

Also, aromatic mono-carboxylic acid used in the present invention is an intermediate generated by the conversion of a corresponding aromatic feedstock compound to aromatic dicarboxylic acid.

Therefore, in the present invention, both an aromatic feedstock compound and a corresponding aromatic mono-carboxylic acid intermediate are introduced and used for an oxidation reaction, so that the aromatic feedstock compound produces a corresponding aromatic mono-carboxylic acid intermediate, and the aromatic mono-carboxylic acid intermediate is again converted into aromatic dicarboxylic acid.

Herein, the aromatic mono-carboxylic acid intermediate is present in liquid phase under the reaction temperature and pressure, and thus, plays a role of a solvent instead of a low-molecular weight carboxylic acid. Also, the aromatic mono-carboxylic acid intermediate is introduced in a large amount, together with the aromatic feedstock compound at the initial stage of the reaction, and thus the concentration is uniformly maintained above a certain level even though there is some variation during the reaction. Accordingly, from the standpoint of the concentration, there is no problem in performing a function as a solvent.

Aromatic mono-carboxylic acid used as a reaction solvent in the present invention is preferably used in an amount of 1˜20 parts by weight based on 1 part by weight of the aromatic feedstock compound. An excessive or insufficient amount (out of the above range) of aromatic mono-carboxylic acid may cause a decrease in the yield of aromatic dicarboxylic acid.

Also, for the reaction solvent, aromatic mono-carboxylic acid is preferably used in an amount of 1 to 20 parts by weight based on 1 part by weight of water. Herein, if the content of aromatic mono-carboxylic acid is less than 1 part by weight of water, an oxidation reaction is not well progressed. On the other hand, if the content of aromatic mono-carboxylic acid is more than 20 parts by weight of water, an oxidation reaction is well progressed, but the cost of raw material is increased. In other words, the use of an excessive amount of aromatic mono-carboxylic acid as a solvent increases the cost for its recovery.

In short, aromatic mono-carboxylic acid plays a role of both a raw material for aromatic dicarboxylic acid and a solvent in an oxidation reaction. From the point of a reaction rate, since the rate of conversion of aromatic feedstock compound into aromatic mono-carboxylic acid is usually higher than that of conversion of aromatic mono-carboxylic acid into aromatic dicarboxylic acid, the amount of aromatic mono-carboxylic acid is increased at an initial stage, and then is returned to its original level as the reaction progresses.

Herein, the reaction time is preferably within the range of 20 to 180 minutes. The reaction is completed when aromatic feedstock compound is exhausted, and the concentration of aromatic mono-carboxylic acid becomes equal to that of an initial stage of the reaction by conversion of aromatic mono-carboxylic acid into aromatic dicarboxylic acid.

After the completion of the reaction, products include aromatic mono-carboxylic acid, of which the amount equals to the introduced amount at an initial stage of the reaction, aromatic dicarboxylic acid, of which the amount equals to the consumed amount of an aromatic feedstock compound introduced to the reaction, and a small amount of aromatic mono-carboxylic aldehyde.

Usually, from among the products, aromatic mono-carboxylic acid has a melting point lower than the temperature of an oxidation reaction, and aromatic dicarboxylic acid has a melting point higher than the temperature of an oxidation reaction. Accordingly, after the completion of the oxidation reaction, only if the temperature is lowered to the level above the melting point of aromatic mono-carboxylic acid and the lowered temperature is maintained, aromatic dicarboxylic acid may be additionally crystallized. Then, aromatic mono-carboxylic acid, and aromatic dicarboxylic acid are separated as liquid phase and solid phase, respectively.

Therefore, through a solid-liquid separation process at the temperature, two kinds of materials are separated and recovered, and then the separated aromatic mono-carboxylic acid in liquid phase is re-used by recycle into a reactor.

With the method like this, it is possible to achieve direct conversion effect of aromatic feedstock compound into aromatic dicarboxylic acid without additional introduction of aromatic mono-carboxylic acid.

In the case of a small amount of aromatic mono-carboxylic aldehyde, which is generated in the reaction, except the amount that is co-precipitated during generation and crystallization of aromatic dicarboxylic acid, most of the amount is attached on the surface of aromatic dicarboxylic acid particles, and is easily dissolved in aromatic mono-carboxylic acid in the molten state.

Especially, even though the melting point of aromatic mono-carboxylic acid is lower than the temperature of an oxidation reaction, its melting point is very high enough to exceed the evaporation point of a low-molecular weight carboxylic acid used as a solvent in a conventional process. Accordingly, lots of impurities generated in an oxidation reaction process can be dissolved in aromatic mono-carboxylic acid in the liquid phase. Therefore, an aromatic dicarboxylic acid cake obtained from a solid-liquid separation process has higher purity than that from a conventional process.

According to the present invention, the reaction temperature of the oxidation reaction is preferably within the range of 150 to 300° C., and the reaction pressure is preferably within the range of 15 to 30 kg/cm² g.

When water and aromatic mono-carboxylic acid are used as a solvent, most of the component vaporized by an exothermic reaction is water because aromatic mono-carboxylic acid has a high boiling point and low volatility.

Heat of vaporization of water is used to remove heat generated by an oxidation reaction, and vaporized water is condensed again by a condenser and is refluxed into a reactor so that the reaction temperature can be regularly maintained.

Herein, although the oxidation reaction produces water, in addition to aromatic mono-carboxylic acid, a certain amount of water is required to be introduced at the initial stage of the reaction so that the exothermic heat of the oxidation reaction can be sufficiently removed by using the heat of vaporization of water.

Since a fairly large amount of water is vaporized by reaction heat of the oxidation reaction, a large part of generated terephthalic acid is crystallized within the reactor. Also, since most of the vaporized component within the reactor is water, in order to regularly control the concentration of water within the reactor, a part of condensates is simply purged to the outside of the reactor. On the other hand, there has been a need to again separate the purged condensates for recovery of solvent in a conventional process. Accordingly, the process based on the invention is simplified. Also, the purged water has relatively high purity, and thus can be re-used for the process.

When air is used as an oxygen source, exhaust gas of the reactor includes nitrogen gas and unreacted oxygen gas, and may also include carbon monoxide and carbon dioxide generated by partial burning of an aromatic feedstock compound.

An unreacted aromatic feedstock compound may be also included in the exhaust gas. Such gas components are not condensed in a condenser, and a part of energy can be recovered by using the gas for expander after recovering unreacted aromatic feedstock compound and other volatile organic compounds.

In the oxidation reaction system according to the present invention, manganese is used as a main catalyst, and a transition metal element is used as a sub catalyst, which is used to activate the catalysis by synergy with manganese, and to increase a reaction yield.

As a precursor for providing manganese, manganese acetate hydrate is preferably used, and as a transition metal element, acetate hydrate or sulfate hydrate is preferably used. Such compounds can dissociate manganese or a transition metal element within a solvent.

In a conventional oxidation reaction system for preparing aromatic dicarboxylic acid by using a low-molecular weight carboxylic acid solvent, it is known that manganese increases the reaction rate by synergy effect with cobalt.

In addition, manganese can bring about catalysis by independently participating in radical generation of a hydrocarbon compound due to high redox potential. Also, depending on kinds of solvents, such activity of manganese is expected to be more excited. Especially, manganese is more economical than cobalt, and thus is used as a main catalyst in the oxidation reaction system of the present invention.

In the conventional oxidation reaction system, bromine, together with cobalt and manganese, has been used as a reaction initiator. Bromine is known to help the catalysis of cobalt by forming a complex with cobalt.

In an oxidation reaction system of the present invention, the use of bromine is basically excluded, and accordingly, manganese, instead of cobalt, is mainly emphasized. Manganese is preferably used in the system in an amount of 1,000 to 10,000 ppm based on the total amount of reactants.

A transition metal element, which is a sub catalyst used in a catalyst system according to the present invention, preferably includes, but is not limited to, one material selected from the group including titanium, zirconium, nickel, chromium, and zinc.

Herein, the content of the transition metal element is preferably included in an amount of 1 to 20% by mole with respect to the manganese. In the catalyst system according to the present invention, the use of an excessive amount of manganese decreases the reactivity because the effect of a sub catalyst is lost, and on the other hand, the use of an excessive amount of the sub catalyst does not improve the reactivity any more, even decreases the reactivity, and is also disadvantageous to variable cost.

Also, in the oxidation reaction system according to the present invention, carbon dioxide is introduced as a reaction stabilizer for high-temperature oxidation reaction, thereby suppressing the excessive combustion of aromatic feedstock compound and the production of by-products such as aromatic mono-carboxylic aldehyde. Accordingly, it is possible to improve reaction selectivity by using carbon dioxide.

When air is used as a source for oxygen molecules, carbon dioxide is preferably introduced in an amount of 5 to 50% as measured by partial pressure within an oxidation reactor. The introduction of an excessive amount (out of the above range) of carbon dioxide decreases the reactivity due to insufficiency of oxygen, and on the other hand, the introduction of an insufficient amount (out of the above range) of carbon dioxide increases the burning of aromatic feedstock compound.

Herein, only a part of carbon dioxide generated by liquid-phase oxidation of aromatic feedstock compound is purged, and the rest of the carbon dioxide is recycled into the inside of the oxidation reactor. Therefore, there is no need to additionally introduce carbon dioxide from the outside.

Especially, since carbon dioxide is reused in the oxidation reactor, the amount of carbon dioxide gas emitted into the atmosphere is decreased, and thus, the effect of suppression of greenhouse gas emission is also expected.

According to an aspect of the present invention, there is provided a method of preparing aromatic dicarboxylic acid, the method including the steps of: preparing a feed mixture by mixing an aromatic feedstock compound, aromatic mono-carboxylic acid, and water in a feed mixture drum (the 1st step); introducing the prepared feed mixture together with air into an oxidation reactor, and carrying out liquid-phase oxidation through agitation (the 2nd step); transferring products of the liquid-phase oxidation to a crystallizer and crystallizing crude aromatic dicarboxylic acid dissolved in liquid-phase aromatic mono-carboxylic acid and water (the 3rd step); obtaining crude aromatic dicarboxylic acid by solid-liquid separation of the crystallized aromatic dicarboxylic acid from the liquid-phase aromatic mono-carboxylic acid and water (the 4th step); and purifying the crude aromatic dicarboxylic acid in a hydrogenation reactor, and obtaining purified aromatic dicarboxylic acid as a solid by solid-liquid separation (the 5th step).

In a preparation process according to the present invention, both the oxidation process and the purification process use water as a solvent, and thus the oxidation process and the purification process can be integrated into single process without sectionalization, unlike a conventional process of aromatic dicarboxylic acid.

The overall process is progressed in the following order: preparation of a feed mixture, an oxidation reaction, crystallization, solid-liquid separation, preparation of crude terephthalic acid slurry, a hydrogenation reaction, crystallization, solid-liquid separation, and drying. Herein, acetic acid, that is, low-molecular weight carboxylic acid, is not used as a solvent, and thus devices that should be used for recovering acetic acid are not required in a conventional system. Also, since a catalyst system excluding bromine is used, there is no need to provide devices for treating bromine, such as a bromine scrubber.

Also, the use of a common solvent for both an oxidation process and a purification process eliminates the need for a dryer and a silo for crude aromatic dicarboxylic acid.

[Best Mode]

Reference will now be made in detail to the preferred embodiments of the present invention. However, the following examples are illustrative only, and the scope of the present invention is not limited thereto.

The following Example 1 and Example 2 show the changes in the yield of terephthalic acid (aromatic dicarboxylic acid) according to the changes in the ratio of manganese and cobalt when water and para-toluic acid (aromatic mono-carboxylic acid) are used as a co-solvent.

### EXAMPLE 1

15 g of para-Xylene (PX), 40 g of para-toluic acid (p-tol), and 20 g of water were added to a reactor, and were mixed to prepare a feed mixture. Mn(CH₃COO)₂.4H₂O or Co(CH₃COO)₂.4H₂O was used as a reaction catalyst in an amount according to Table 1. Then, at 190° C. under pressure of 2.5 MPa, air was continuously introduced at a flow rate of 0.5 L/min to perform an oxidation reaction.

The yields of terephthalic acid were obtained as noted in Table 1.

TABLE 1

| Manganese | Cobalt acetate | Reaction | Yield of terephthalic |
|---|---|---|---|
| acetate (g) | (g) | time (min) | acid (mol %)¹ |
| 0.10 | 0.10 | 90² | 29.1 |
| 0.20 | 0.10 | 185 | 113.4 |
| 0.20 | 0.07 | 250 | 134.0 |
| 0.27 | 0.07 | 250 | 129.8 |
| 0.35 | 0.07 | 180 | 130.1 |

¹Calculated on PX
²The reaction was automatically stopped

### EXAMPLE 2

An oxidation reaction was performed in the same manner as described in Example 1, except that 40 g of water, instead of 20 g of water, was used, and catalysts were introduced in an amount according to Table 2.

The yields of terephthalic acid were obtained as noted in Table 2.

TABLE 2

| Manganese | Cobalt | Reaction time | Yield of terephthalic |
|---|---|---|---|
| acetate (g) | acetate (g) | (min) | acid (mol %)¹ |
| 0.40 | — | 180 | 124.4 |
| 0.30 | — | 90² | 30.4 |
| 0.30 | 0.15 | 90² | 24.7 |
| 0.25 | 0.15 | 90² | 31.4 |
| 0.20 | 0.15 | 90² | 28.1 |
| 0.15 | 0.15 | 80² | 26.9 |

¹Calculated on PX
²The reaction was automatically stopped

In Example 1 and Example 2, 20 g of water and 40 g of water were used, respectively. In both Examples, the increase in the amount of manganese and the decrease in the amount of cobalt resulted in increase of a reaction yield. Especially, when a large amount of water is introduced, the yield of the reaction can be significantly increased only if cobalt is not introduced.

The following Example 3 shows the changes in the yield of terephthalic acid (aromatic dicarboxylic acid) according to manganese and the kind of a sub-catalyst when water and para-toluic acid (aromatic mono-carboxylic acid) are used as a co-solvent.

### EXAMPLE 3

8 g of para-Xylene (PX), 15.3 g of para-toluic acid (p-tol), and 6.5 g of water were added to a reactor, and were mixed to prepare a feed mixture. 0.23 g of Mn(CH₃COO)₂.4H₂O together with a sub catalyst as noted in Table 3 were used as reaction catalysts. Then, at 195° C. under pressure of 2.5 MPa, air was continuously introduced at a flow rate of 0.180 L/min to perform an oxidation reaction.

As noted in Table 3, when titanium was introduced as a sub catalyst, the reaction yield was the highest, and on the other hand, when iron was introduced, the reaction yield was decreased.

TABLE 3

| | Amount of | Yield of terephthalic |
|---|---|---|
| Sub catalyst | introduction (g) | acid (wt %)¹ |
| Not introduced | 0.02 | 80.7 |
| Ni-acetate | 0.02 | 82.7 |
| Zr-acetate | 0.02 | 84.2 |
| Ti-sulfate | 0.02 | 90.3 |
| Fe-acetate | 0.02 | 74.2 |
| Cr-acetate | 0.02 | 81.1 |

¹Calculated on PX and p-tol

The following Example 4 shows the effect of the amount of CO₂ introduced on the changes in the yield of terephthalic acid (aromatic dicarboxylic acid) and the selectivity, when water and para-toluic acid (aromatic mono-carboxylic acid) are used as a co-solvent, and also manganese and a titanium sulfate compound (as a sub catalyst) are used for reaction catalysts.

### EXAMPLE 4

An oxidation reaction was performed by using Ti(SO₄)₂ as a sub-catalyst in the same manner as described in Example 3, except that CO₂ was introduced as a reaction stabilizer as noted in Table 4.

If we consider the changes in the reaction yield, the amount of 4-CBA(4-carboxybenzaldehyde) (a by-product) produced, and the amount of PX burning depending on the amount of CO₂ introduced, as noted in Table 4, the use of 400 ml of CO₂ resulted in the highest yield and selectivity. On the other hand, when an excessive or insufficient amount of CO₂ was introduced, the yield and selectivity was decreased.

Also, as compared to the case where CO₂ was not introduced, the introduction of CO₂ can result in higher reactivity.

TABLE 4

| Introduced | | | |
|---|---|---|---|
| amount | Yield of terephthalic | Production of | Combustion |
| of CO₂ (mL) | acid (wt %)¹ | 4-CBA (wt %) | of PX (wt %) |
| 0 | 90.3 | 4.2 | 3.2 |
| 200 | 92.1 | 3.9 | 2.8 |
| 400 | 94.2 | 3.2 | 2.2 |
| 600 | 93.4 | 3.5 | 2.1 |

¹Calculated on PX and p-tol

### INDUSTRIAL APPLICABILITY

As can be seen from the foregoing, in a method of preparing aromatic dicarboxylic acid according to the present invention, by using a mixture of aromatic mono-carboxylic acid and water is used as a co-solvent, and a mixture of manganese and a small amount of transition metal element is used as catalysts, and carbon dioxide is used as a reaction stabilizer. As a result, the yield acceptable for a commercial scale was obtained. In addition, the exclusion of acetic acid, bromine, and cobalt significantly decreases both capital cost and variable cost, and fundamentally removes harmfulness to a workplace. Also, it is possible to suppress the emission of greenhouse gases by recycling carbon dioxide generated by burning of a raw material into an oxidation reactor.

1. A method of preparing aromatic dicarboxylic acid, the method comprising:
an oxidation process of preparing crude aromatic dicarboxylic acid by liquid-phase oxidation of an aromatic feedstock compound; and
a purifying process of removing impurities by hydrogenation of the crude aromatic dicarboxylic acid,
wherein the oxidation process uses a mixed solvent comprising water and aromatic mono-carboxylic acid as a reaction solvent.

2. The method as claimed in claim 1, wherein the aromatic mono-carboxylic acid used as the reaction solvent is used in an amount of 1 to 20 parts by weight based on 1 part by weight of the aromatic feedstock compound.

3. The method as claimed in claim 1, wherein in the reaction solvent, the aromatic mono-carboxylic acid is mixed in an amount of 1 to 20 parts by weight based on 1 part by weight of water.

4. The method as claimed in claim 1, wherein the aromatic mono-carboxylic acid corresponds to the aromatic feedstock compound, and is a compound selected from the group including a compound represented by Formula 1 and a compound represented by Formula 2: 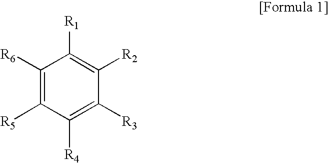
wherein, from R₁ to R₆, one substituent is an alkyl group selected from the group including methyl, ethyl and isopropyl, another substituent is a carboxylic group, and the other substituents are hydrogen; and 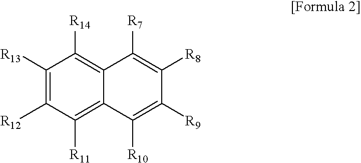
wherein, from R₇ to R₁₄, one substituent is an alkyl group selected from the group including methyl, ethyl and isopropyl, another substituent is a carboxylic group, and the other substituents are hydrogen.

5. The method as claimed in claim 1, wherein the oxidation process uses a catalyst system comprising manganese and a transition metal element, as reaction catalysts.

6. The method as claimed in claim 5, wherein the manganese is used in an amount of 1,000 to 10,000 ppm based on the total amount of reactants.

7. The method as claimed in claim 5, wherein the transition metal element is included in an amount of 1 to 20% by mole with respect to the manganese.

8. The method as claimed in claim 6, wherein the transition metal element is one material selected from the group including titanium, zirconium, nickel, chromium, and zinc.

9. The method as claimed in claim 1, wherein the oxidation process uses carbon dioxide as a reaction stabilizer.

10. The method as claimed in claim 9, wherein carbon dioxide generated by liquid-phase oxidation of the aromatic feedstock compound is reused.

11. The method as claimed in claim 9, wherein, when air is used as a source for oxygen molecules, the carbon dioxide is introduced in an amount of 5 to 50% as measured by partial pressure within an oxidation reactor.

12. The method as claimed in claim 1, wherein the oxidation process is performed at 150 to 300° C.

13. The method as claimed in claim 1, wherein the oxidation process is performed under pressure of 15 to 30 kg/cm² g.

14. The method as claimed in claim 1, wherein the oxidation process is performed for 20 to 180 minutes.

15. A method of preparing aromatic dicarboxylic acid, the method comprising the steps of:
preparing a feed mixture by mixing an aromatic feedstock compound, aromatic mono-carboxylic acid, and water in a feed mixture drum;
introducing the prepared feed mixture together with air into an oxidation reactor, and carrying out liquid-phase oxidation through agitation;
transferring products resulted from the liquid-phase oxidation to a crystallizer, and crystallizing crude aromatic dicarboxylic acid dissolved in liquid-phase aromatic mono-carboxylic acid and water;
obtaining crude aromatic dicarboxylic acid as solid by solid-liquid separation of the crystallized aromatic dicarboxylic acid from the liquid-phase aromatic mono-carboxylic acid and water; and
purifying the crude aromatic dicarboxylic acid through introduction into a hydrogenation reactor, and obtaining purified aromatic dicarboxylic acid after solid-liquid separation.

16. The method as claimed in claim 15, which is dispensed with an additional devices for recovering acetic acid and removing bromine.

17. The method as claimed in claim 2, wherein in the reaction solvent, the aromatic mono-carboxylic acid is mixed in an amount of 1 to 20 parts by weight based on 1 part by weight of water.

18. The method as claimed in claim 7, wherein the transition metal element is one material selected from the group including titanium, zirconium, nickel, chromium, and zinc.

19. The method as claimed in claim 10, wherein, when air is used as a source for oxygen molecules, the carbon dioxide is introduced in an amount of 5 to 50% as measured by partial pressure within an oxidation reactor.
